(19) United States
(12) Patent Application Publication
(10) Pub. No.: US 2026/0360462 A1
(43) Pub. Date: Nov. 12, 2026

(54) PROCESS ANALYSIS USING ENVIRONMENTAL SEQUENCE DATA TO DETERMINE THE TAXONOMY OF ORGANISMS PRESENT IN AN ENVIRONMENT

(71) Applicants: Robert K. Lowery, Port St. Lucie, FL (US); Tyler G. Serio, Knoxville, TN (US)

(72) Inventors: Robert K. Lowery, Port St. Lucie, FL (US); Tyler G. Serio, Knoxville, TN (US)

(21) Appl. No.: 19/667,052

(22) Filed: May 4, 2026

Related U.S. Application Data
(60) Provisional application No. 63/801,719, filed on May 7, 2025.

(51) Int. Cl. G16B 10/00; G16B 30/10; G16B 45/00; G16B 50/00

(52) U.S. Cl. CPC G16B 10/00; G16B 30/10; G16B 45/00; G16B 50/00

(57) ABSTRACT

A method of automated analysis of whole-genomic sequences from environmental samples is disclosed. The method provides a networked pipeline that automates sequence processing, from initial filtering to taxonomic visualization. Technical features include length-based filtering to isolate ancient DNA, and a damage-mitigation step that modifies quality scores for terminal thymine pairs to prevent the loss of degraded sequences. The pipeline transforms sequences from FASTQ to FASTA formats and executes high-speed alignment searches against genomic databases. Taxonomic ambiguity is resolved using a lowest common taxonomic rank logic. Output includes an interactive, hierarchical sunburst chart representing the relative proportions of identified organisms. The method includes a download interface allowing users to retrieve specific sequences and alignments, such as SAM or BAM files, directly from the hierarchical visualization. The invention enables the efficient processing of voluminous nuclear DNA data while accounting for characteristic ancient DNA damage patterns.

### CROSS-REFERENCE TO RELATED APPLICATIONS

This application claims priority under 35 USC § 119(c) of U.S. Provisional Application Ser. No. 63/801,719, filed on May 7, 2025, and entitled “PROCESS ANALYSIS USING ENVIRONMENTAL SEQUENCE DATA TO DETERMINE THE TAXONOMY OF ORGANISMS PRESENT IN AN ENVIRONMENT,” the entire disclosure of which is incorporated herein by reference.

### TECHNICAL FIELD

The present invention relates generally to the field of bioinformatics and metagenomics, and more specifically to automated computer-implemented pipelines for the taxonomic identification and analysis of whole-genomic DNA from environmental samples.

### BACKGROUND OF THE INVENTION

Organisms continually contribute genetic material to their surroundings, creating environmental DNA (eDNA) reservoirs in soil, water, and air. Analyzing eDNA provides critical insights into the biodiversity of a locale, both historical and contemporary, and is of significant value in fields ranging from ecology to forensics.

Currently, the most common method for inferring the species present in an environmental sample is “Metabarcoding” (or “Barcoding”). This technique relies on the targeted amplification of specific sequences found in mitochondrial or chloroplast chromosomes. Because these organellar chromosomes exist in high copy numbers per cell. (ranging from hundreds to thousands), they offer a high probability of detection.

However, Metabarcoding suffers from significant technical and informational limitations. By design, it ignores the nuclear genome, which constitutes over 99% of an organism's genetic data and contains the vast majority of its functional genes. Furthermore, because nuclear DNA accounts for roughly three times the gross volume of DNA in a cell compared to organellar DNA, researchers who rely solely on Metabarcoding have a higher statistical likelihood of missing organisms present in low concentrations.

Despite the clear advantages of analyzing whole genomic DNA, the practice remains underutilized due to several technical barriers:

Data Volume and Complexity: Whole-genomic data is prodigious and requires significant processing power and time (often days or weeks) to analyze.

Lack of Accessibility: Existing data pipelines for whole-genomic alignment are notoriously user-unfriendly and remain the specialized domain of bioinformaticians. Publicly available resources have focused almost exclusively on the simpler, less computationally intensive Metabarcoding datasets.

DNA Degradation: Environmental DNA is typically highly fragmented and degraded. Current automated pipelines often fail to account for the specific chemical damage patterns (such as terminal deamination) characteristic of ancient DNA, leading to the premature filtering and loss of valuable data.

There exists a need in the art for an automated, streamlined, and accessible data pipeline capable of processing whole genomic environmental DNA, which does not ignore Eukaryotic organisms such as Plants, Animals and Fungi. Such a method must be able to account for DNA degradation patterns, resolve taxonomic ambiguities efficiently, and provide a user-friendly interface for researchers to access and download alignment results without requiring specialized bioinformatics expertise.

### SUMMARY OF THE INVENTION

The present invention, known as the Bailey Pipeline, provides a computer-implemented system and method for the automated, high-throughput analysis of metagenomic sequence data. The invention solves technical problems inherent in bioinformatics—specifically the difficulty of isolating and accurately identifying degraded “ancient” DNA (aDNA) from environmental samples—by providing a seamless, networked pipeline that automates the operation and data transfer between a specific sequence of genomic executables.

In a first embodiment, the invention provides a technical workflow optimized for identifying potential ancient DNA fragments within a genomic input file. The process begins by categorizing and segregating groups of input reads based on a maximum length threshold, as shorter reads are characteristic of ancient DNA degradation. For data integrity, duplicate reads are removed so that they are not counted or analyzed twice,

The method identifies terminal thymine pairs at the front or end of each read. The pipeline adjusts quality scores based on these pairs to prevent ancient damage from being penalized during subsequent quality control operations. Following this modification, a SolexaOA++ executable performs quality control by programmatically removing any read with a combined quality score below a predetermined quality score threshold. In one embodiment, the predetermined quality score threshold is 40. The data is then transformed from a FASTQ format to a FASTA format by stripping metadata (empty space and quality score lines) to enable high-speed Megablast alignment searching.

The embodiment resolves taxonomic ambiguity by condensing reads with multiple, equally probable database matches into a single lowest common taxonomic rank (LCTR). The final output is an interactive Krona sunburst chart that displays the relative proportions of identified organisms in a taxonomic hierarchy, allowing researchers to download the specific sequences and alignments that produced the matches shown in the chart.

In a second embodiment, the invention provides a flexible pipeline that allows users to prioritize either ancient DNA, modern DNA, or a combination of both within an environmental sample, This embodiment utilizes a PEAR (Paired-end Read Merger) (J. Zhang, K. Kobert, T. Flouri, A. Stamatakis. PEAR: A fast and accurate Illumina Paired-End reAd mergeR. Bioinformatics 30(5):614-620, 2014) executable to pair forward and reverse FASTQ reads.

For ancient DNA prioritization, the method executes a custom script that programmatically sets Phil's Read Editor (PHRED) scores for thymine bases at the first, second, last, and next-to-last positions of each paired strand to a value of 2. This targeted modification prevents the premature removal of usable ancient sequences during filtering. The method then applies a multi-stage filtering process: first removing sequences with a quality score less than 15, and subsequently using SolexaQA (Solexa Quality Assessment) (Cox, M. P. Peterson, D. A., and Biggs, P. J., “SolexaQA: At-a-glance quality assessment of Illumina second-generation sequencing data,” BMC Bioinformatics, Vol. 11, No. 485, Sep. 27, 2010) to remove sequences with a score below 40.

Following deduplication and conversion to FASTA format, the pipeline performs a Megablast search against a GenBank nucleotide database. A best-matching alignment is resolved for each hit, assigning a taxonomic rank as either a species or a more inclusive taxon ranging from Genus to Domain. Using accession codes, the method retrieves full taxonomic lineage from the National Center for Biotechnology Information (NCBI) database to produce an interactive, hierarchical Krona pie chart. This visualization serves as a functional interface, allowing users to select specific taxa to download the corresponding sequences and alignments in SAM or BAM file formats.

### Technical Advantages

(1) The Bailey Pipeline provides a way for individuals who have one to millions of sequenced whole-genomic DNA reads from a biological or environmental source to find out what organisms, including Eukaryotes such as Plants, Animals and Fungi contributed their DNA to that sample, without being limited to only examining mitochondrial reads, as is done with conventional Metabarcoding techniques used for similar purposes. As current Metabarcode based pipelines do not sufficiently support the analysis whole genomic DNA of Plants, Animals and Fungi (as well as many other Eukaryotic organisms), the Bailey Pipeline provides a major technical advantage in that no other pipeline exists for such data, which is readily available to the public.
(2) Additionally, by providing a “pipeline” for processing of user DNA reads that provides final results in a user-friendly way, without the user having to spend endless hours managing the output and input of numerous executables, the Bailey Pipeline provides a significant convenience advantage to the highly technical and arduous process of having to learn to code Python and bioinformatic theory to find out what organisms are in their DNA. samples,
(3) The Bailey Pipeline also allows for the isolation and inclusion of ancient DNA sequences which are often found in environmental samples at some level. This optional inclusion of older DNA sequences may be of interest for persons for whom acquiring ancient DNA or knowing the relative age of a particular organism's DNA is of interest.

Across both embodiments, the Bailey Pipeline improves the field of bioinformatics by:
(1) Providing a DNA sequence Pipeline to process whole-genomic DNA and assess not only the Prokaryotic organisms that contribute to a biological/environmental sample, but also the Eukaryotic organisms such as Plants, Animals and Fungi.
(2) Making the bioinformatic technique of DNA analysis from biological samples using whole genomic DNA more available to more user groups which do not have access to specialized bioinformatic teams, which are usually high-cost and frequently inaccessible to most interested users. By increasing the use of DNA analysis by more users, the field of bioinformatics should grow the number of active researchers and participants, which may improve the field by creating a greater demand for user-friendly bioinformatic programs which are easily accessible.
(3) Automating tool integration: Seamlessly stringing together open-source executables into a functional, networked pipeline.
(4) Mitigating DNA damage bias: Utilizing specific thymine-based quality score modifications to retain degraded ancient fragments that would otherwise be discarded by standard filters.
(5) Resolving Taxonomic Ambiguity: Implementing a mathematical approach to condense multiple probable hits into the lowest common taxonomic rank, ensuring higher accuracy in metagenomic reporting.

### BRIEF DESCRIPTION OF THE DRAWINGS

FIG. 1 is an interactive, hierarchical sunburst pie chart representing the taxonomic distribution of organisms identified within a sample, according to one or more embodiments of the invention.

### DETAILED DESCRIPTION OF THE PREFERRED EMBODIMENTS

The present invention relates to a computer-implemented method and system, referred to herein as the “Bailey Pipeline” or “Bailey Protocol,” for the automated taxonomic identification and analysis of genomic sequences. The method is specifically configured to handle environmental DNA (eDNA) and degraded ancient DNA (aDNA) by Implementing a sequence of automated data transformations that mitigate the technical challenges associated with degraded genetic material.

In a first embodiment, the pipeline is configured as a multi-step automated workflow. The process begins by categorizing and segregating groups of input reads based on a maximum length threshold, as shorter reads are characteristic of ancient DNA degradation. Because ancient DNA is typically highly fragmented, this step isolates the shorter sequences most likely to be of ancient origin. Following the segregating by size, the method performs duplicate removal to eliminate redundant reads, ensuring each unique sequence is analyzed only once to prevent statistical bias.

The pipeline then performs a damage-masking modification. The method programmatically identifies terminal thymine pairs at the front (5′ end) or back (3′ end) of each read, Because cytosine-to-thymine deamination is a hallmark of ancient DNA damage, the method adjusts the quality scores for these specific thymine pairs. This prevents the pipeline from discarding a read simply because it exhibits characteristic ancient damage.

Following this modification, the method executes a quality control operation using a SolexaQA++ executable. In this embodiment, any read with a combined quality score below a predetermined quality score threshold is programmatically discarded. In one embodiment, the predetermined quality score threshold is 40. The remaining high-confidence reads are transformed from a FASTQ format to a FASTA format by removing the metadata lines (the empty space line and the quality score line). The lengths of these FASTA sequences are counted and recorded before being passed to a Megablast alignment search.

The method assigns database IDs to each read based on match probability, To resolve taxonomic ambiguity, the method implements a Lowest Common Taxonomic Rank (LCTR) logic. If a read matches multiple organisms with equal probability, the method assigns the read to the most specific taxonomic rank shared by all those organisms, The results are then formatted for visualization in a hierarchical sunburst chart.

In a second embodiment, the pipeline allows for the targeted prioritization of ancient or modem DNA. Input FASTQ files are paired using a PEAR executable. For ancient DNA prioritization, a custom script modifies the PHRED quality scores for thymine bases at the first, second, last, and next-to-last positions of each paired strand, setting them to a value of 2.

The sequences undergo a multi-stage filtering process: first removing reads with a quality score below 15, then removing reads below 40 using SolexaQA. After deduplication and conversion to FASTA, the sequences are aligned using Megablast against the GenBank database. Taxonomic resolution is performed by assigning a rank as either a species or a more inclusive taxon ranging from Genus to Domain based on alignment scores. Accession codes are then used to retrieve full lineage information from an NCBI database.

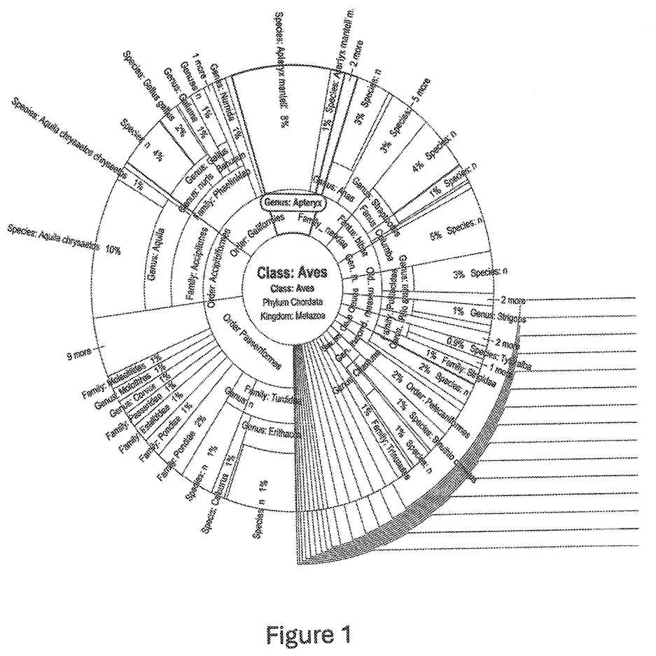

FIG. 1 illustrates an output of the Bailey Pipeline in the form of an interactive, hierarchical sunburst pie chart. This visualization provides a graphical representation of the taxonomic distribution of the organisms found within a sample. The chart shown in FIG. 1 is a non-limiting example of a taxonomic distribution for a single sample. The hierarchical sunburst pie chart may be adapted to represent various datasets, classification levels, or sample types without departing from the scope of the present disclosure

### Hierarchical Structure and Concentric Rings:

The chart is organized in a taxonomic hierarchical way, characterized by a series of concentric rings radiating outward from a central point. The innermost circle represents the highest and most inclusive taxonomic rank. As shown in FIG. 1, the center identifies the broad biological (Linnaean) classification of the sample, such as Kingdom: Metazoa, Phylum: Chordata, and Class: Aves.

Each subsequent ring moving outward represents a progressively more specific taxonomic level:
The first outer ring identifies the Order (e.g., Passeriformes, Galliformes),
The next ring identifies the Family (e.g., Phasianidae, Accipitridae).
The following ring identifies the Genus (e.g., Apteryx, Gallus, Aquila).
The outermost segments identify the Species level (e.g., Gallus gallus, Aquila chrysaetos).

### Quantitative Representation (Relative Proportions):

The angular width (the “slice” of the pie) for any given segment is proportional to the number of sequences assigned to that specific taxon. For example, FIG. 1 displays a significant segment for ‘Species: Apteryx mantelli 8%’, indicating that 8% of the total sequences were assigned to that specific species within the Apteryx genus. This allows a researcher to see at a glance which organisms are most prevalent in the environmental sample.

### Taxonomic Resolution and Ambiguity:

The chart visually demonstrates the Lowest Common Taxonomic Rank (LCTR) logic. Where a sequence cannot be confidently assigned to a specific species but is confirmed at a higher level, the segment terminates at the corresponding ring (e.g., “Genus: n” or “Family: n”). This ensures that the visualization is an accurate reflection of the genomic evidence without over-assigning ambiguous reads.

### Functional and Interactive Download Interface:

The chart serves as a functional interface for data retrieval. Each taxonomic name: in the chart is a “clickable” element. Upon selecting a name (for example, clicking on “Aquila”), the method provides a download interface. This allows the user to download a file containing the specific set of sequences and alignments (such as SAM or BAM files) that led to that organism being represented in the chart. Consequently, the drawing represents not just a static image, but a gateway to the raw genomic data and the specific alignments (Best Hits) generated during the pipeline's execution.

It will be understood that the content, hierarchy, and resolution of the chart may vary significantly depending on the sample composition. For example, while FIG. 1 shows a broad distribution, the pipeline may generate a visualization focused on Genus and species levels if a sample is dominated by a single genus, or any other subset of taxonomic data appropriate to the input.

The present invention has been described in terms of specific embodiments incorporating details to facilitate the understanding of the principles of construction and operation of the invention. As such, references herein to specific embodiments and details thereof are not intended to limit the scope of the claims appended hereto. It will be apparent to those skilled in the art that modifications can be made in the embodiments chosen for illustration without departing from the spirit and scope of the invention.

We claim:

1. A method of automated genomic sequence analysis, comprising:
a. categorizing and segregating groups of input reads based on a maximum length threshold;
b. removing duplicate reads from the segregated groups of input reads;
c. modifying quality scores of the segregated groups of input reads by identifying thymine pairs at the front or end of each read and adjusting quality scores based on the thymine pairs to prevent ancient damage from being counted against a sequence;
d. performing quality control on the modified reads by programmatically removing any read having a combined quality score below a predetermined quality score threshold;
e. converting the modified reads from a FASTQ format to a FASTA format by removing two lines per read consisting of empty space and a quality score to generate FASTA sequences;
f. counting and recording the lengths of the FASTA sequences;
g. running a Megablast alignment search on the FASTA sequences against a genomic database to generate hits and identify a plurality of organisms having best matches to the input reads;
h. assigning database IDs to each FASTA sequence based on the highest likelihood of belonging to those sequences;
i. matching the database IDs to taxonomic IDs and relating them back to the FASTA sequences using original sequence IDs from the input reads;
j. retrieving taxonomic lineage information for the hits from a taxonomic database;
k. condensing FASTA sequences associated with multiple equally probable database IDs to a single lowest common taxonomic rank;
l. formatting the taxonomic information and lowest common taxonomic rank results into a Krona-compatible format; and
m. producing an interactive Krona sunburst chart representing the organisms having best matches, wherein the sunburst chart displays relative proportions of the organisms organized in a taxonomic hierarchy.

2. The method of claim 1, wherein the categorizing and segregating further comprises separating the input reads into a plurality of different groups based on predefined length ranges.

3. The method of claim 1, wherein the performing quality control is executed using a SolexaQA++ executable.

4. The method of claim 1, wherein condensing the FASTA sequences to the lowest common taxonomic rank comprises identifying a taxonomic rank shared by all organisms associated with the multiple equally probable database IDs for a specific FASTA sequence.

5. The method of claim 1, wherein the taxonomic hierarchy of the Krona sunburst chart includes rings representing the biological classification levels of Kingdom, Phylum, Class, Order, Family, Genus, and Species.

6. The method of claim 1, wherein matching the database IDs to taxonomic IDs includes correlating the taxonomic information back to the original sequence IDs from the input reads to maintain the identity of the sequences throughout the analysis.

7. The method of claim 1, further comprising providing a downloadable file containing the specific FASTA sequences or alignments that produced the best matches represented in the Krona sunburst chart.

8. The method of claim 1, wherein the modifying quality scores based on thymine pairs performed prior to the performing quality control to retain usable ancient DNA.

9. The method of claim 1, wherein the predetermined quality score threshold is 40.

10. A method of automated genomic sequence analysis, comprising:
a. receiving input genomic files as FASTQ files comprising forward and reverse reads;
b. receiving a user selection to prioritize a sequence subset comprising ancient DNA, modem DNA, or both;
c. pairing the forward and reverse reads to one another using a PEAR executable;
d. modifying quality scores for sequences within the ancient DNA subset by programmatically setting PHRED scores for thymine bases located at the first, second, last, and next-to-last positions of each paired strand to a value of 2;
e. filtering the sequences by removing any sequence having a quality score less than 15;
f. executing a SolexaQA quality control operation on the remaining sequences to remove any sequence having a quality score below 40;
g. removing duplicate reads from the sequences remaining after the SolexaQA quality control operation;
h. converting the sequences from a FASTQ format to a FASTA format;
i. performing a Megablast alignment search of the FASTA sequences against a GenBank nucleotide database to generate hit tables and determine a plurality of organisms found within each sample;
j. resolving a best-matching alignment for each hit, wherein a taxonomic rank is assigned to each hit as either a species or a more inclusive taxon ranging from Genus to Domain;
k. retrieving taxonomic identifiers and taxonomic lineage information for the best-matching alignments using accession codes from the hit tables;
l. generating a taxonomy file and producing an interactive Krona pie chart representing the organisms found within each sample, wherein the pie chart displays the organisms in a taxonomic hierarchy; and
m. providing a download interface where a user can select a taxon name from the interactive Krona pie chart to download the specific sequences and alignments that resulted in that taxon being represented.

11. The method of claim 10, wherein the alignments available for download via the download interface are provided in a SAM or BAM file format.

12. The method of claim 10, wherein the step of retrieving taxonomic lineage information comprises cross-referencing the accession codes against an NCBI taxonomy database.

13. The method of claim 10, wherein the modification of quality scores to a PHRED score of 2 is performed only when the user selection prioritizes the sequence subset comprising ancient DNA.

14. The method of claim 10, wherein the modification of quality scores to a PHRED score of 2 is omitted if the user selection prioritizes only modern DNA.

15. The method of claim 10, wherein the step of resolving a best-matching alignment includes calculating scores produced by Megablast to identify the most probable taxonomic origin even when a singular species match is unavailable.

16. The method of claim 10, wherein the taxonomic hierarchy of the interactive Krona pie chart comprises concentric rings representing Kingdom, Phylum, Class, Order, Family, Genus, and Species.

17. The method of claim 10, wherein the names of taxa from the interactive Krona pie chart are selected by a user to trigger the download of all sequences that produced the set of best hits leading to the representation of that taxon.

18. The method of claim 10, wherein the modification of the PHRED scores for the thymine bases is performed by a custom script to retain usable ancient DNA.

19. The method of claim 10, wherein the taxonomy file is generated by a custom script that uses accession codes for best hits to retrieve taxonomic IDs and lineage.
